(19) United States
(12) Patent Application Publication
(10) Pub. No.: US 2025/0229265 A1
(43) Pub. Date: Jul. 17, 2025

(54) FUNCTIONAL ELEMENT, BETA CONTAINER, TRANSFER SYSTEM AND BARRIER SYSTEM

(71) Applicant: Groninger & Co. GmbH, Crailsheim (DE)

(72) Inventors: Denis BORKMANN, Satteldorf (DE); Armin MERZ, Ellwangen (DE)

(21) Appl. No.: 19/095,715

(22) Filed: Mar. 31, 2025

Related U.S. Application Data
(63) Continuation of application No. PCT/EP2023/077625, filed on Oct. 5, 2023.

(30) Foreign Application Priority Data
Oct. 7, 2022 (DE) 10 2022 125 968.4

(51) Int. Cl. B01L 1/02; B65B 3/00; B65B 31/02; C12M 1/34

(52) U.S. Cl. CPC B01L 1/02; B65B 3/003; B65B 31/02; C12M 41/36; B01L 2200/026; B01L 2200/0689; B01L 2300/041

(57) ABSTRACT

A functional element for a beta container of a transfer system, wherein the functional element is arrangeable on and/or in a beta port opening of a beta port, and the functional element comprises a functional element opening that is smaller than the beta port opening. There is also disclosed a functional element for a beta container of a transfer system, wherein the functional element is arrangeable on and/or in a beta port opening of a beta port, the functional element comprises one or more nutrient medium carrier holders, and each nutrient medium carrier holder is configured to hold a nutrient medium carrier. Furthermore, there is also disclosed a beta container for a transfer system, a transfer system, and a barrier system, such as an isolator system.

### CROSS-REFERENCE TO RELATED APPLICATIONS

This is a Continuation application of International patent application PCT/EP2023/077625, filed Oct. 5, 2023, which claims the priority of German patent application DE 10 2022 125 968.4, filed Oct. 7, 2022. The whole content of both applications is herein incorporated by reference.

### FIELD

The present invention generally relates to a functional element for a beta container of a transfer system, to a beta container of a transfer system comprising such a functional element, to a transfer system comprising such a beta container, and to a barrier system, in particular an isolator system, comprising such a transfer system.

### BACKGROUND

A barrier system is understood to be a system that provides a physical and aerodynamic barrier, for example by means of positive air pressure, between an external environment, such as an external cleanroom environment, and a working process. Various barrier systems are known in the prior art. A barrier system can, for example, be an isolator or a restricted access barrier system (RABS). The RABS can be an open RABS or a closed RABS.

The present disclosure primarily concerns aseptic isolators as barrier systems. However, the present disclosure can also be applied in other barrier systems.

The term “isolator” is generally understood to mean a container that is hermetically and gas-tight sealed from the surrounding workspace. Within an isolator, a defined atmosphere can be created for processing sensitive or hazardous products.

In this context, isolators are typically used in biopharmaceutical process technology, for example as part of a filling system with multiple process and processing stations, to create a highly pure or sterile, i.e., germ-free, environment.

In such filling systems, containers, in particular vials, cartridges, bottles, syringes, and/or the like, can be filled with a product, preferably a pharmaceutical or cosmetic product, particularly a liquid or powder, and then sealed with a closure element, such as a stopper or a closure cap, particularly a crimp cap.

Pharmaceutical filling systems are generally located in a low-germ environment. In particular, a germ-free environment must be maintained in the filling area within the isolator. This condition is monitored by placing nutrient medium carriers, also called microbial collectors, at critical points. Nutrient medium carriers can, for example, be Petri dishes that contain a nutrient medium. When a microbe comes into contact with the nutrient medium, the microbe grows during subsequent incubation, allowing contamination to be retrospectively detected. Such monitoring of a germ-free environment is referred to as “microbial monitoring” or “microbiological monitoring.”

Transfer systems can be used for transferring objects, such as closure elements or nutrient medium carriers, into or out of the isolator. A transfer system can comprise a transfer lock that is couplable to the isolator from the outside. Objects can be transferred into or out of the isolator via the transfer lock. A transfer system can, for example, be configured as a port system, in particular as an alpha-beta port system. Such transfer systems comprise an alpha port and a beta container. The beta container serves as a transfer lock. The beta container comprises a beta port. The alpha port is preferably arranged on an isolator wall of the isolator, which encloses an internal space of the isolator. The alpha port and the beta port are couplable to connect the internal space of the isolator with an internal volume of the beta container. In the coupled state, objects can be introduced from the beta container into the isolator or removed from the isolator into the beta container.

Feed devices can be used to supply closure elements that are provided in a beta container. A feed device can be arranged inside the isolator and positioned on the alpha port from the inside. The feed device can, for example, comprise a chute or a tube. The closure elements can then be introduced or supplied into the isolator from the beta container via the feed devices.

Such feed devices are known in the prior art.

For example, document DE 10 2021 101 384 B3 discloses a system for transporting sterile bulk closure elements from an environment of an isolator into an internal space of the isolator, comprising a container for storing a supply quantity of closure elements in the environment of the isolator, an isolator opening, and a collection device for collecting the closure elements and providing the closure elements in the internal space of the isolator, with a metering device for controlling a target quantity of closure elements transported from the container through the isolator opening into the collection device.

Furthermore, document EP 3 581 339 B1 discloses a transfer system for a sealed enclosure, wherein the sealed enclosure defines a first closed volume and comprises at least one sealed connection device intended to connect the first closed volume with a second closed volume, wherein the transfer system is intended to be arranged in the enclosure, wherein the transfer system comprises at least one arm intended to be rotatably mounted on a wall of the sealed enclosure via a first rotary joint having a first rotational axis, wherein the transfer system comprises a chute having a docking edge and a discharge edge, wherein the docking edge is designed to interact with the sealed connection device, and wherein the transfer system comprises a second rotary joint between the arm and the chute, wherein the second rotary joint has a second rotational axis.

A port system can also be used for supplying or removing nutrient medium carriers into or out of an isolator. The nutrient medium carriers can, in particular, be provided in a beta container. A handling device can be provided inside the isolator, for example, to remove the nutrient medium carriers from the beta container or return them to it. Alternatively, the nutrient medium carriers can also be manually removed from or returned to the beta container using glove ports.

For example, document DE 10 2020 102 758 B4 discloses a method for automated microbial monitoring in an isolator, wherein the isolator comprises a transfer lock, and the method comprises the following steps: first providing at least one nutrient medium carrier holder at a first position inside the isolator; second providing at least one nutrient medium carrier inside the transfer lock; first robot-assisted transferring of each individual nutrient medium carrier of the at least one nutrient medium carrier from the transfer lock to a free nutrient medium carrier holder of the at least one nutrient medium carrier holder; and first robot-assisted arranging of the transferred nutrient medium carrier in the free nutrient medium carrier holder. Furthermore, this document discloses a system for automated microbial monitoring in an isolator and a computer program.

However, the known transfer systems still allow room for improvements, particularly with regard to structural design, object transfer, and operational safety.

### SUMMARY

Against this background, it is an object of the present disclosure to improve the structural design of a transfer system and a barrier system. Furthermore, it is an object of the present disclosure to improve the transfer of objects in a transfer system and a barrier system. Furthermore, it is an object of the present disclosure to improve the operational safety of a transfer system and a barrier system.

According to a first aspect, a functional element for a beta container of a transfer system is provided, wherein the functional element is arrangeable on and/or in a beta port opening of a beta port of the beta container, wherein the functional element comprises a functional element opening, wherein the functional element opening is smaller than the beta port opening.

According to a second aspect, a functional element for a beta container of a transfer system is provided, wherein the functional element is arrangeable on and/or in a beta port opening of a beta port of the beta container, wherein the functional element comprises one or more nutrient medium carrier holders, wherein each nutrient medium carrier holder is configured to hold a nutrient medium carrier.

According to a third aspect, a beta container for a transfer system is provided, wherein the beta container comprises an internal volume, an outer wall enclosing the internal volume, a beta port that is couplable with an alpha port of the transfer system, and the functional element according to the first or second aspect.

According to a fourth aspect, a transfer system is provided, wherein the transfer system comprises an alpha port and the beta container according to the third aspect.

According to a fifth aspect, a barrier system is provided, wherein the barrier system comprises the transfer system according to the fourth aspect. The barrier system is, in particular, an isolator system comprising an isolator.

The isolator can comprise an internal space. The isolator can comprise an isolator wall that encloses or surrounds the internal space. The isolator wall separates the internal space from an external environment surrounding the isolator. The isolator is preferably an aseptic isolator. An aseptic isolator provides a highly pure or sterile, i.e., germ-free, environment in the internal space.

The isolator can preferably be part of a filling system with multiple process and processing stations. The filling system can, for example, be a system for filling and sealing containers with a pharmaceutical or cosmetic substance. The system can, in particular, comprise a filling station and at least one sealing station.

The transfer system is used for transferring objects, in particular closure elements or nutrient medium carriers, into or out of the isolator. For this purpose, the transfer system comprises the alpha port and the beta container.

The alpha port can be arranged on the isolator wall of the isolator. The alpha port comprises an alpha port opening. The alpha port opening forms a passage through the isolator wall. In other words, the isolator is accessible from the outside through the alpha port opening. The alpha port opening can have any shape. For example, the alpha port opening can be circular, elliptical, or rectangular. In a preferred embodiment, the alpha port opening is circular.

Furthermore, the alpha port can comprise an alpha port base body. The alpha port base body is preferably ring-shaped. In particular, the alpha port base body can extend through the isolator wall. The alpha port base body comprises an inner side and an outer side. The inner side faces the internal space. The outer side faces the external environment. The outer side is, in particular, a side of the alpha port facing the beta port. The alpha port base body can comprise the alpha port opening. In particular, the alpha port opening can be formed by a recess in the alpha port base body, which extends from the inner side to the outer side of the alpha port base body. In particular, the alpha port base body surrounds the alpha port opening. The alpha port can comprise an alpha port flange on the outer side. In particular, the alpha port base body can form the alpha port flange on the outer side. The alpha port flange surrounds the alpha port opening.

Furthermore, the alpha port can comprise a door. The door is arranged at the alpha port opening of the alpha port. The door serves to open and close the alpha port opening. Preferably, the door is movably, in particular pivotably, arranged on the alpha port base body of the alpha port. The door can be moved to open or close the alpha port opening. In a closed state, the door seals the alpha port opening tightly. To move the door, the alpha port can, for example, comprise a drive mechanism.

The beta container comprises an internal volume. Objects, such as closure elements or nutrient medium carriers, can be arranged in the internal volume. The beta container can comprise an outer wall. The outer wall surrounds or encloses the internal volume. In particular, the outer wall isolates the internal volume from an external environment surrounding the beta container. The outer wall can, for example, be designed to be flexible. Specifically, the outer wall can be a flexible bag. The flexible bag can be made of plastic. The flexible bag can, for example, be a sterile bag. Alternatively, the outer wall can be rigid. In particular, the outer wall can be designed as a rigid housing. The housing can be made of plastic or metal, such as aluminum or stainless steel.

The beta container comprises the beta port. The beta port can be arranged on the outer wall of the beta container. The beta port comprises a beta port opening. The beta port opening forms a passage through the outer wall. In other words, the internal volume is accessible from the outside through the beta port opening. The beta port opening can have any shape. For example, the beta port opening can be circular, elliptical, or rectangular. In a preferred embodiment, the beta port opening is circular.

Furthermore, the beta port can comprise a beta port base body. The beta port base body is preferably ring-shaped. In particular, the beta port base body can extend through the outer wall. The beta port base body comprises an inner side and an outer side. The inner side faces the internal volume. The outer side faces the external environment. The outer side is, in particular, a side of the beta port facing the alpha port. The beta port base body can comprise the beta port opening. In particular, the beta port opening can be formed by a recess in the beta port base body, which extends from the inner side to the outer side of the beta port base body. In particular, the beta port base body surrounds the beta port opening. The beta port can comprise a beta port flange on the outer side. In particular, the beta port base body can form the beta port flange on the outer side. The flange surrounds the beta port opening.

Furthermore, the beta port can comprise a cover element. The cover element is arrangeable at or in the beta port opening of the beta port. The cover element serves to cover or seal the beta port opening. The cover element can be detachably couplable with the beta port base body or the beta port flange. When the cover element is coupled with the beta port base body or the beta port flange, the cover element tightly seals the beta port opening and completely covers it.

The alpha port is couplable with the beta port. In a coupled state, the internal volume of the beta container is connected to the internal space of the isolator. In the coupled state, the alpha port and the beta port are coupled in such a way that the internal volume and the internal space are isolated from the external environment. In particular, a seal can be arranged between the alpha port and the beta port. In the coupled state, the beta port flange can be arranged on the alpha port flange. Specifically, the seal can be arranged between the alpha port flange and the beta port flange.

The beta port opening and the alpha port opening are preferably of equal size. In a preferred embodiment, the beta port opening and the alpha port opening are circular. In particular, the beta port opening and the alpha port opening can have the same diameter.

For coupling, the alpha port and the beta port can, for example, comprise corresponding coupling elements, wherein one or more coupling elements of the alpha port are couplable with one or more coupling elements of the beta port. The coupling elements can be arranged on the alpha port base body and the beta port base body or on the beta port flange and the alpha port flange

The door and the cover element can also be couplable. Only when the door and the cover element are coupled can they be moved together. In particular, they can be moved in such a way that the alpha port opening and the beta port opening can be opened and closed together. Specifically, the door and the cover element can couple with each other when the alpha port and the beta port are coupled. This allows the alpha port opening and the beta port opening to be opened and closed together when the alpha port and the beta port are coupled.

For coupling, the door and the cover element can, for example, comprise corresponding coupling elements, wherein one or more coupling elements of the door are couplable with one or more coupling elements of the cover element. The coupling elements can, for example, form a bayonet lock.

Objects such as closure elements or nutrient medium carriers can be provided in the beta container, which are to be transferred into the isolator. In the coupled state of the alpha port and the beta port, the objects can be transferred or introduced into the isolator. Furthermore, objects can also be transferred or introduced from the isolator into the beta container in the coupled state to transfer these objects out of the isolator.

When objects are transferred from the beta container into the isolator or from the isolator into the beta container in the coupled state of the alpha and beta ports, the objects are transferred through the alpha port opening and the beta port opening in a transfer direction. The transfer direction can, in particular, be parallel to a direction extending from the inner side to the outer side of the beta port.

According to the disclosure, an additional functional element is provided for the beta container. The functional element is preferably arranged in the beta container. The functional element serves to improve the transfer of objects between the beta container and the isolator. In particular, the functional element serves to improve the transfer of objects from the beta container into the isolator and/or the transfer of objects from the isolator into the beta container.

The functional element is arrangeable on and/or in the beta port opening. Preferably, the functional element can be at least partially arranged in the beta port opening. Alternatively, the functional element can also be arranged in the internal volume of the beta container adjacent to the beta port opening. The functional element can be arranged on the beta port base body of the beta port. In particular, the functional element can be, preferably detachably, fastened to the beta port base body. Alternatively, the beta port base body can also form the functional element.

The functional element can comprise a functional element body. The functional element body can comprise an outer edge. The outer edge can be arranged on the beta port base body. In particular, the outer edge can rest circumferentially against the beta port base body in the beta port opening when the functional element is at least partially arranged in the beta port opening

The functional element according to the first aspect also comprises the functional element opening. Regarding the functional element opening, a radial direction, an axial direction, and a tangential direction, also referred to as the circumferential direction, can be defined. The radial direction is a direction extending radially outward from the opening, in particular from a center of the functional element opening. The axial direction is a direction in which the functional element opening extends through the functional element. The circumferential direction is a direction that runs around the functional element opening. The radial direction, axial direction, and circumferential direction are perpendicular to each other. The functional element is preferably arrangeable in the beta container in such a way that the axial direction runs parallel to the transfer direction of the objects.

The functional element opening is smaller than the beta port opening. In particular, a cross-sectional geometry of the functional element opening perpendicular to the axial direction is smaller than a cross-sectional geometry of the beta port opening perpendicular to a direction from the outer side to the inner side. Specifically, a diameter of the functional element opening is smaller than a diameter of the beta port opening.

The functional element body of the functional element according to the first aspect can extend outward in the radial direction from the functional element opening. The functional element opening can, in particular, be designed as a recess in the functional element body. The functional element according to the first aspect can comprise a functional element flange surrounding the functional element opening. Specifically, the functional element body can comprise or form the functional element flange.

The functional element according to the first aspect can comprise a first axial end and a second axial end in the axial direction, which are arranged on opposite sides of the functional element. The functional element flange can be arranged at the first axial end. The functional element flange surrounds the functional element opening at the first axial end. The functional element can be arranged in the beta container in such a way that the first axial end faces away from the internal volume and is directed toward the alpha port. Accordingly, the functional element can be arranged in the beta container in such a way that the second axial end faces the internal volume and is directed away from the alpha port.

Objects, such as closure elements, can be provided in the beta container, particularly in its internal volume. When the beta port is coupled with the alpha port, these objects can then be introduced into the isolator. For this purpose, they must be transferred through the beta port opening and the alpha port opening. The functional element is arranged in such a way that the objects are transferred through the functional element opening on their transfer path from the internal volume into the isolator. The functional element according to the first aspect is particularly suitable for closure elements as the objects to be transferred.

In a beta container without a functional element, the discharge cross-section of these objects corresponds to the size of the beta port, in particular to the cross-sectional geometry of the beta port. The term “discharge cross-section” refers to the cross-sectional area available for the objects as they pass through the beta port opening, perpendicular to the transfer direction. The functional element opening serves to reduce this discharge cross-section. In other words, in a beta container with a functional element according to the first aspect, the discharge cross-section is reduced. In particular, this means that the cross-sectional area of the discharge cross-section is spaced apart from the edge of the beta port opening.

During the transfer of objects in a transfer system between a beta container and an isolator, it is known that contact of the objects to be transferred with the so-called “ring of concern” should be avoided. The “ring of concern” includes, for example, the contact surfaces between the alpha port and the beta port, in particular a circumferential line of the seal between the alpha port and the beta port. It is particularly desirable to largely or completely avoid contact of the objects with the beta port opening and the alpha port opening.

For example, in the prior art, when using sterile bags as beta containers, an internal hose has been used, which, in the coupled state of the alpha and beta ports, can be pulled out of the sterile bag to bridge the “ring of concern.” However, the hose must be pulled out of the sterile bag from within the isolator using glove ports. This requires an additional manual operation, which can lead to the potential transfer of germs via the glove.

By providing the functional element according to the first aspect in the beta container, the “ring of concern” is also bridged. As previously explained, the functional element opening reduces the discharge cross-section and, in particular, spaces it apart from the edge of the beta port opening. This also allows contact of the objects with the beta port opening and the alpha port opening, particularly with the “ring of concern,” to be largely or completely avoided. The functional element according to the first aspect thus bridges the “ring of concern” without requiring manual operation.

In beta containers with a rigid housing made of, for example, stainless steel, the “ring of concern” has so far been bridged in the prior art by a specific arrangement of the alpha port and the feed device. As a feed device, a chute or a pivoting tube was provided within the isolator at the alpha port, which could preferably be introduced, in particular pivoted, into the alpha port opening automatically to bridge the “ring of concern.” However, it was previously necessary to arrange the alpha port at an angle so that the tube could be pivoted in the direction of gravity and the circular openings of the alpha port and the tube were concentrically aligned. For this purpose, it was particularly required to provide a bay on a vertical isolator wall, which allowed the port to be arranged at an angle to the isolator wall. The bay increased the manufacturing costs of the isolator. Moreover, the bay created a critical point for vortex formation in relation to the laminar airflow inside the isolator. Furthermore, the bay also made cleaning more difficult due to additional corners and edges.

By providing the functional element according to the first aspect in the beta container, this specific arrangement of the alpha port is no longer necessary. If a feed device, such as a tube, is inserted at an angle into the alpha port, the entry cross-section of the feed device is reduced and is thus no longer adapted to the alpha port opening. In the case of a cylindrical tube as a feed device, inserting it at an angle can result in an elliptical entry cross-section.

In beta containers without a functional element, this leads to objects passing through the beta port opening and the alpha port opening not necessarily being transferred into the feed device but instead passing through the openings beside the feed device. As a result, they may come into contact with the “ring of concern,” fall onto the machine, or become jammed.

By providing the functional element according to the first aspect in the beta container, it can be prevented that objects move past the feed device within the openings of the alpha port and the beta port. This is particularly achieved because the functional element also reduces the discharge cross-section from the beta port opening. In particular, the functional element opening can be designed so that the discharge cross-section is smaller than or equal to the entry cross-section.

The functional element according to the second aspect comprises one or more nutrient medium carrier holders. Each nutrient medium carrier holder can be configured to hold a nutrient medium carrier. Specifically, each nutrient medium carrier holder can accommodate a nutrient medium carrier. The functional element according to the second aspect thus serves to provide one or more nutrient medium carriers in the beta container.

The one or more nutrient medium carrier holders can be arranged on the functional element body. Each nutrient medium carrier holder can, for example, comprise a receiving element or a support surface for holding a respective nutrient medium carrier. The nutrient medium carrier holders are preferably arranged on a side of the functional element facing the alpha port.

The functional element according to the second aspect can thus be used to transfer one or more nutrient medium carriers into or out of the isolator.

The isolator system can comprise a handling device inside the isolator, which is configured to handle the nutrient medium carriers. The handling device can, in particular, be designed as a handling robot. The handling device can, for example, comprise a multi-axis arm and an end effector arranged at one end of the arm. The end effector can comprise a gripping tool, preferably a gripper. Using the gripping tool, nutrient medium carriers can be grasped and transferred.

The handling device can be specifically configured to grasp a nutrient medium carrier arranged in one of the nutrient medium carrier holders of the functional element and transfer it to a position inside the isolator. The handling device can also be configured to grasp a nutrient medium carrier positioned inside the isolator and transfer it to one of the nutrient medium carrier holders of the functional element. Such a handling device for transferring nutrient medium carriers is described, for example, in document DE 10 2020 102 758 B4 of the applicant. In particular, with an isolator system designed in this way, microbial monitoring can be carried out as described in document DE 10 2020 102 758 B4 of the applicant.

Using the functional element according to the second aspect, nutrient medium carriers can thus be provided or arranged in the beta container simply and safely.

In a first refinement of the aspects, the functional element opening can be circular.

Preferably, the beta port opening and, in particular, the alpha port opening can also be circular. Specifically, the diameter of the functional element opening is smaller than the diameter of the beta port opening. The diameter of the functional element opening can be 20% to 80%, preferably 35% to 65%, and particularly 50% of the diameter of the beta port opening.

In another refinement of the aspects, the functional element opening can be arrangeable concentrically to the beta port opening.

Preferably, the functional element opening and the beta port opening have the same shape, with the beta port opening being larger than the functional element opening. The term “concentric” is understood here to mean that the functional element opening and the beta port opening are symmetrically arranged about a central axis.

In another refinement of the aspects, the functional element can be ring-shaped or funnel-shaped.

In the case of a funnel shape, both sides of the funnel are open. The functional element opening is formed by the inner recess of the ring shape or funnel shape. The outer edge of the ring can be arranged circumferentially in the beta port opening. In this way, the functional element can be properly fitted into the beta port opening.

In another refinement of the aspects, the functional element can be designed as an insert element that is insertable into the beta container, in particular into the beta port opening.

In this way, existing beta containers that previously did not have a functional element can be suitably retrofitted by inserting the functional element into them. When the functional element is inserted into the beta container, the beta container can, in particular, be sterilized together with the functional element.

In another refinement of the aspects, the functional element can comprise a functional element body, wherein the functional element body comprises an outer edge, wherein the functional element body surrounds the functional element opening, or one or more nutrient medium carrier holders are arranged on the functional element body.

As previously described, the functional element opening can be designed as a recess in the functional element body that extends through the functional element body. Alternatively, one or more nutrient medium carrier holders can be arranged on the functional element body. In particular, the functional element body can support or form one or more nutrient medium carrier holders.

In another refinement of the aspects, the functional element body can comprise an outer edge, wherein the outer edge is elastically formed, in particular in a lamellar manner

In particular, a plurality of lamellae can be arranged circumferentially on the outer edge. If inner protrusions are provided on the beta port base body in the beta port opening, for example as coupling elements for the cover element or the functional element, the elastic design allows the functional element to still be inserted into the beta port opening. The elastic outer edge can simply yield inward at the inner protrusions. After the functional element has passed the inner protrusions during insertion, the outer edge automatically springs back outward.

In another refinement of the aspects, the functional element body can comprise an outer edge, wherein the outer edge is shaped complementary to the beta port opening.

In particular, the outer edge can be arranged circumferentially in the beta port opening on the beta port base body or rest circumferentially against it. In this way, the functional element is fitted into the beta port opening. Objects transferred between the isolator and the internal volume must therefore pass through the functional element opening. Specifically, the outer edge can be radially outward in the radial direction. The outer edge can preferably be arranged at the second axial end.

In another refinement of the aspects, the functional element can comprise one or more fastening elements, by means of which the functional element can be fastened to the beta port, in particular to a beta port base body of the beta port.

In this way, the functional element can be securely connected to the beta port, particularly in an inserted state. This ensures that the functional element is arranged and held in a defined position relative to the beta port, facilitating the transfer of objects from the beta container into the isolator or from the isolator into the beta container. In particular, the functional element can be detachably fastened to the beta port base body using the fastening elements. The beta port base body can comprise corresponding receiving elements. The fastening elements can engage with the receiving elements to fasten the functional element. Each fastening element can engage with a corresponding receiving element. The fastening elements can, in particular, be hook-shaped. Preferably, the fastening elements are arranged on the outer edge of the functional element body.

In another refinement of the aspects, the beta port can comprise the functional element, in particular wherein a beta port base body of the beta port and the functional element are integrally formed.

In other words, the beta port base body and the functional element can be integrally molded. In this refinement, the functional element is thus integrated into the beta container. A beta container that comprises such an integrated functional element is particularly suitable for multiple uses.

In another refinement of the aspects, the beta port can comprise a cover element for covering the beta port opening, wherein the functional element is arranged between the cover element and the internal volume.

The cover element can be used to seal the beta port opening. The functional element can preferably be arranged on or adjacent to the cover element. The cover element thus serves to tightly cover or seal the beta port opening. In particular, the functional element flange can be arranged adjacent to the cover element. This ensures that the functional element opening is positioned as close as possible to an outer end of the beta port opening. In this way, it is ensured that objects to be transferred from the beta container do not come into contact with the “ring of concern.”

In another refinement of the aspects, the beta port can comprise a sealing element for sealing the coupling between the alpha port and the beta port.

The sealing element can preferably be arranged on the outer side of the beta port base body. Specifically, the sealing element can be arranged on the beta port flange. In particular, the beta port base body can comprise a circumferential groove on the outer side around the beta port opening, into which the sealing element is inserted. The sealing element can, for example, be a lip seal. In the coupled state, the sealing element can be arranged between the alpha port flange and the beta port flange. In this way, the internal volume of the beta container and the internal space of the isolator are sealed against an external environment of the isolator.

In another refinement of the aspects, the isolator can comprise an internal space and an isolator wall enclosing the internal space, wherein the alpha port is arranged on the isolator wall, wherein the beta port is couplable with the alpha port from an external environment of the isolator.

When the alpha port and the beta port are coupled, objects can be transferred between the beta container and the isolator.

In another refinement of the aspects, the alpha port can be arranged on a vertical wall surface of the isolator wall.

The vertical wall surface extends in the isolator parallel to a vertical direction. As previously described, the alpha port can be arranged on a vertical wall due to the functional element. Thus, the alpha port does not need to be arranged on a bay.

In another refinement of the aspects, the barrier system can further comprise a feed device in the internal space of the isolator, wherein the feed device is arrangeable on the alpha port from the inside.

The feed device serves to supply objects, in particular closure elements, into the isolator. When the feed device is arranged on the alpha port, the objects can be transferred from the beta container through the alpha and beta ports via the feed device into the isolator. The feed device can comprise a tube or a chute for this purpose. The feed device can also include an arm that is pivotably arranged on the isolator wall, preferably above the alpha port.

In another refinement of the aspects, the feed device can comprise a tube, wherein a first open end of the tube is arrangeable on the alpha port, in particular wherein the tube is cylindrical.

Specifically, the first end of the tube can extend into the alpha port opening. Objects can then be introduced into the isolator from the beta container via the tube. The tube has a first open end and a second open end. When being transferred, the objects enter the tube at the first open end and exit at the second open end.

In another refinement of the aspects, the tube of the feed device can be arrangeable on the alpha port such that, in the coupled state, it extends through an alpha port opening of the alpha port up to the beta port opening, in particular wherein the first end of the tube is arrangeable on a functional element flange of the functional element.

This ensures that objects passing through the functional element opening from the internal volume of the beta container enter directly into the tube of the feed device. This prevents the objects from coming into contact with the “ring of concern.”

In another refinement of the aspects, the tube of the feed device can be arrangeable on the alpha port such that the tube extends at an angle to a horizontal direction, in particular wherein the first end of the tube has an elliptical shape.

Specifically, in this configuration, the second end of the tube is positioned lower in the vertical direction than the first end of the tube. This allows the objects to be moved through the tube by gravity. If the alpha port is arranged on a vertical wall surface, the alpha port opening is oriented horizontally. In other words, in this case, the transfer direction is parallel to a horizontal direction. To ensure that the first end of the tube can still be arranged flush with the functional element flange, the first end of the tube can be cut at an angle accordingly. This results in the first end of the tube being elliptical in shape. In particular, the diameter of the functional element opening is equal to or smaller than the smallest diameter of the elliptical shape of the first end of the tube.

It is understood that the features mentioned above and those still to be explained below can be used not only in the specified combinations but also in other combinations or individually, without departing from the scope of the present invention.

### DRAWINGS

Exemplary embodiments are illustrated in the drawings and are explained in greater detail in the following description. The figures show:

FIG. 1 is an isometric view of a first embodiment of an isolator system;

FIG. 2 is a longitudinal sectional view of the isolator system from FIG. 1;

FIG. 3 is an isometric view of the isolator system from FIG. 1 with a pivoted-away feed device;

FIG. 4 is an isometric view of a transfer system of the isolator system from FIG. 1;

FIG. 5 is an isometric view of a rear side of the transfer system from FIG. 4;

FIG. 6 is a longitudinal sectional view of the transfer system from FIG. 4;

FIG. 7 is an isometric view of an alpha port of the transfer system from FIG. 4;

FIG. 8 is an isometric view of a rear side of the alpha port from FIG. 7;

FIG. 9 is an isometric view of a beta container of the transfer system from FIG. 4;

FIG. 10 is a longitudinal sectional view of the beta container from FIG. 9;

FIG. 11 is an isometric view of the beta container from FIG. 9 without a cover element;

FIG. 12 is an isometric view of a functional element of the beta container from FIG. 9;

FIG. 13 is an isometric view of a rear side of the functional element from FIG. 12;

FIG. 14 is a longitudinal sectional view of the functional element from FIG. 12;

FIG. 15 is an isometric view of a second embodiment of an isolator system;

FIG. 16 is a longitudinal sectional view of the isolator system from FIG. 15;

FIG. 17 is an isometric view of a transfer system of the isolator system from FIG. 15;

FIG. 18 is an isometric view of a beta container of the transfer system from FIG. 17;

FIG. 19 is an isometric view of a rear side of the beta container from FIG. 18;

FIG. 20 is a longitudinal sectional view of the beta container from FIG. 18;

FIG. 21 is an isometric view of a third embodiment of an isolator system;

FIG. 22 is an isometric view of a transfer system of the isolator system from FIG. 21;

FIG. 23 is an isometric view of a rear side of the transfer system from FIG. 22;

FIG. 24 is an isometric view of a beta container of the transfer system from FIG. 22;

FIG. 25 is an isometric view of a rear side of the beta container from FIG. 24;

FIG. 26 is an isometric view of the beta container from FIG. 24 without a cover element;

FIG. 27 is an isometric view of a functional element of the beta container from FIG. 24;

FIG. 28 is an isometric view of a rear side of the functional element from FIG. 27; and

FIG. 29 is a longitudinal sectional view of the functional element from FIG. 27.

### DETAILED DESCRIPTION

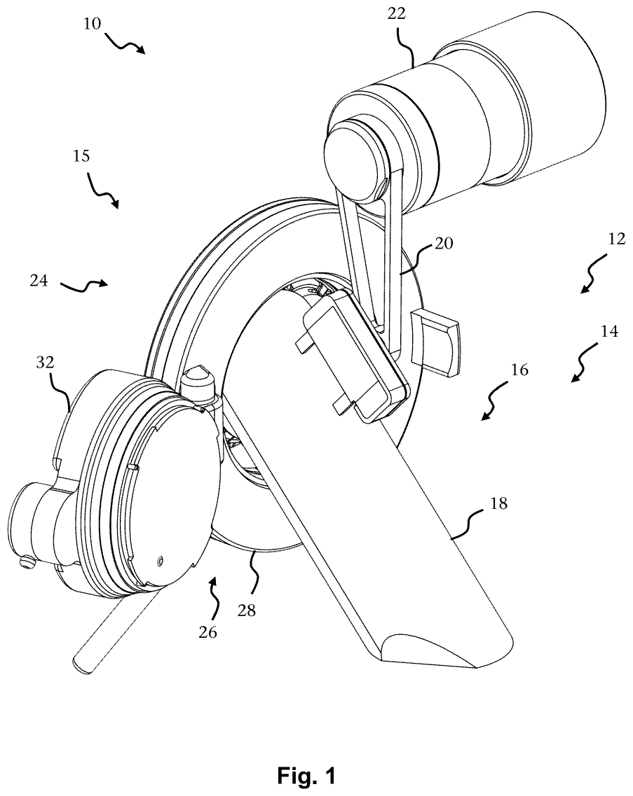

FIGS. 1 to 3 show a first embodiment of an isolator system, designated as barrier system 10 in its entirety.

The isolator system 10 comprises an isolator 12. The isolator 12 can include an internal space 14. The isolator can have an isolator wall (not shown) that encloses or surrounds the internal space 14. The isolator wall separates the internal space 14 from an external environment 15 surrounding the isolator 12. The isolator 12 is preferably an aseptic isolator.

The isolator system 10 can further include a feed device 16. The feed device 16 serves to supply objects into the internal space of the isolator 12. The feed device 16 is arranged inside the internal space 14 of the isolator 12. The feed device 16 comprises a tube 18. It also includes an arm 20, where a first end of the arm 20 is fixedly connected to the tube 18. A second end of the arm 20 is pivotably mounted around a horizontal axis on the isolator wall. The feed device 16 includes a drive mechanism 22, which is configured to pivot the arm 20 along with the tube 18 around the horizontal axis. The tube 18 has a first open end 82 and a second open end 84. The tube 18 extends from the first end 82 to the second end 84 and is substantially cylindrical. The first end 82 of the tube 18 is cut at an angle, making it elliptical in shape. The tube 18 can also be cut at an angle at both ends 82 and 84, making both ends elliptical.

The isolator system 10 further comprises a transfer system 24. The transfer system 24 is used to transfer objects into the isolator and to transfer objects out of the isolator. In particular, the transfer system 24 can be used to introduce objects such as closure elements into the isolator.

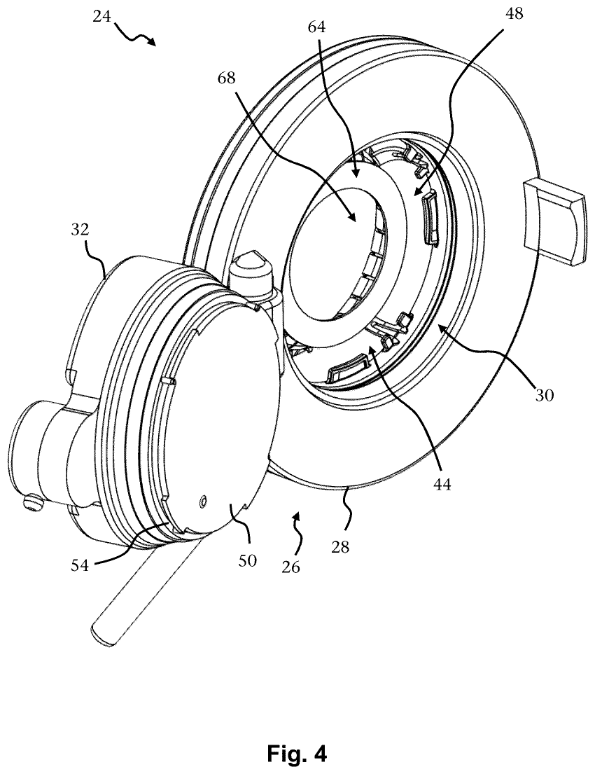

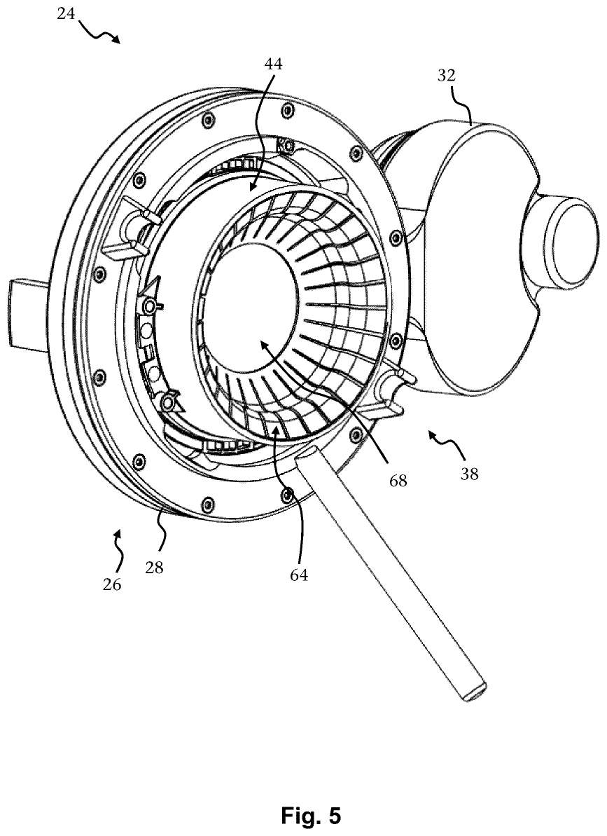

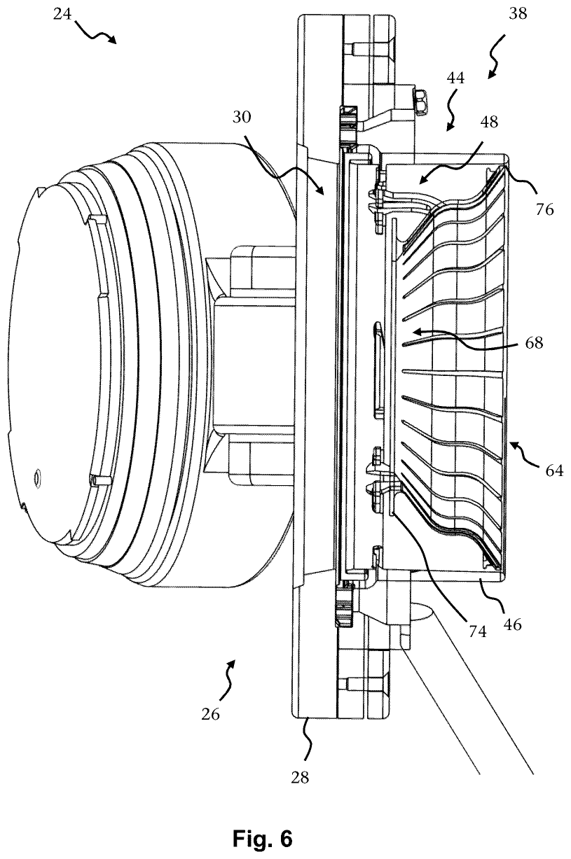

The transfer system 24 is detailed in FIGS. 4 to 6. The transfer system includes an alpha port 26 and a beta container 38. The alpha port 26 can be arranged on the isolator wall of the isolator 12. Specifically, the alpha port 26 is arranged on a vertical wall surface of the isolator wall. The alpha port 26 forms a sealable passage through the isolator wall from the internal space 14 to the external environment 15.

The feed device 16 is arrangeable on the alpha port 26 from the inside. When the feed device 16 is arranged on the alpha port 26, objects can be transferred from the beta container 38 into the isolator 12 via the feed device 16.

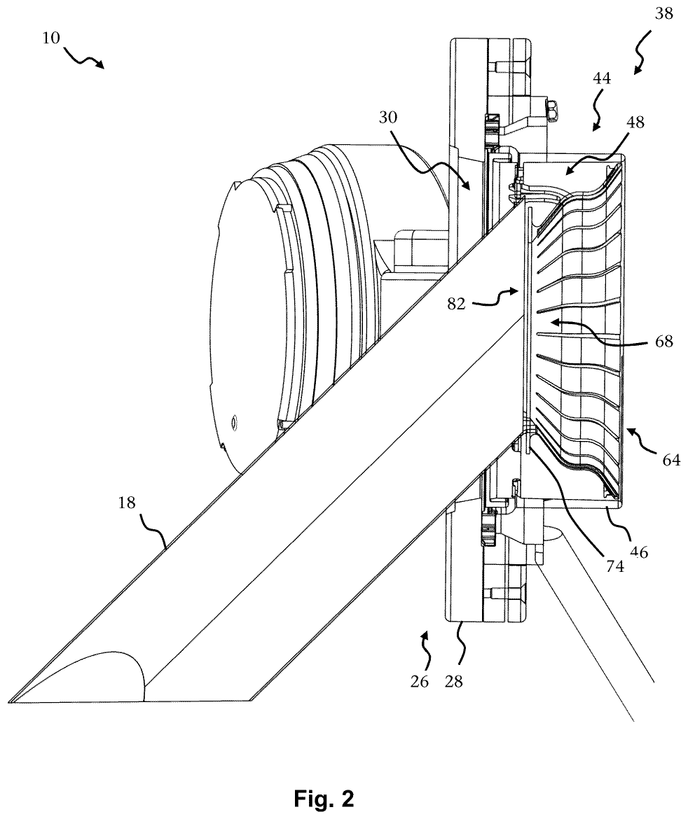

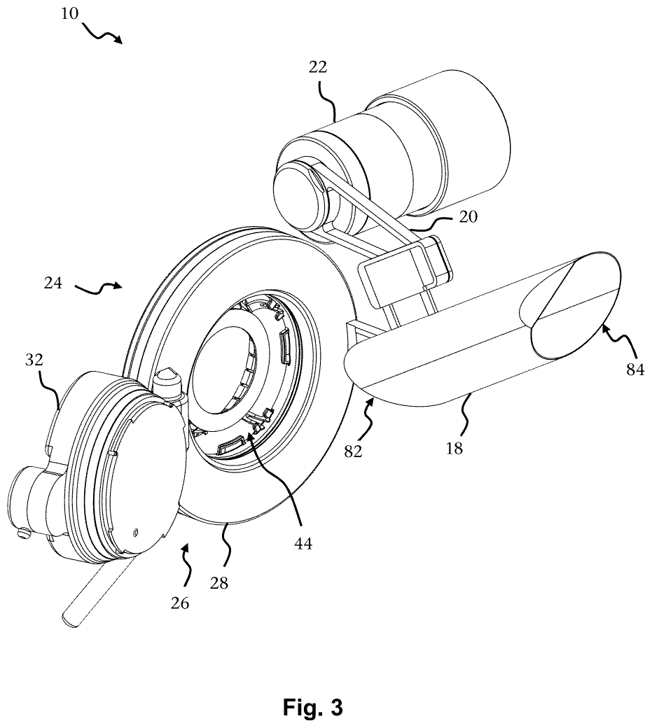

The feed device 16 is preferably pivotably arranged in a vertical direction above the alpha port 26. The tube 18 is pivotable via the arm 20 between a first position and a second position. In FIGS. 1 and 2, the tube 18 is shown in the first position. In the first position, the first end 82 of the tube 18 is arranged at or inside the alpha port 26. When the tube 18 is in the first position, objects from the beta container 38 can be introduced into the tube 18. In FIG. 3, the tube 18 is shown in the second position. In the second position, the first end 82 of the tube 18 is not arranged at the alpha port 26 but is moved away from it and positioned at a distance from the alpha port 26. When the tube 18 is in the second position, the door 32 can be opened and closed.

In the first position of the tube 18, the first end 82 of the tube 18 is positioned higher in the vertical direction than the second end 84. As a result, the tube 18 is inclined relative to a horizontal direction.

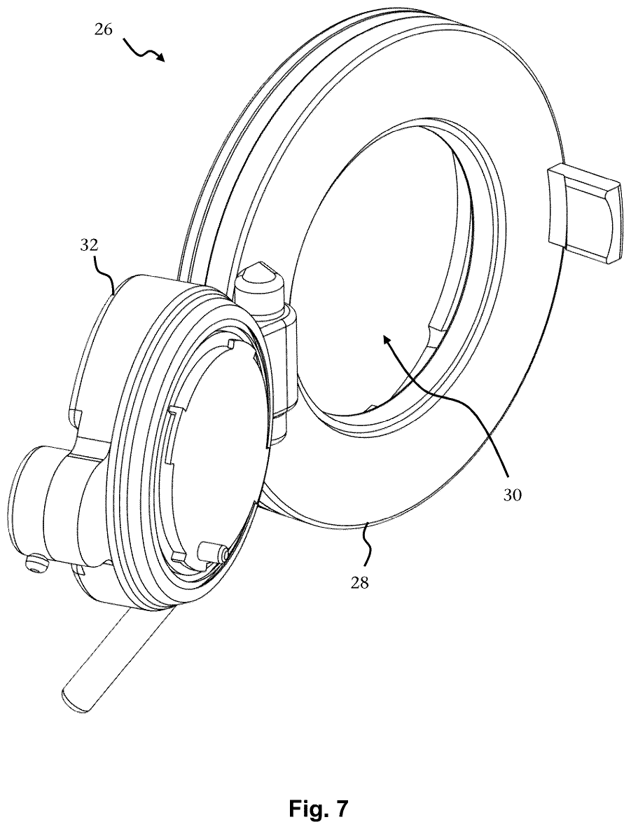

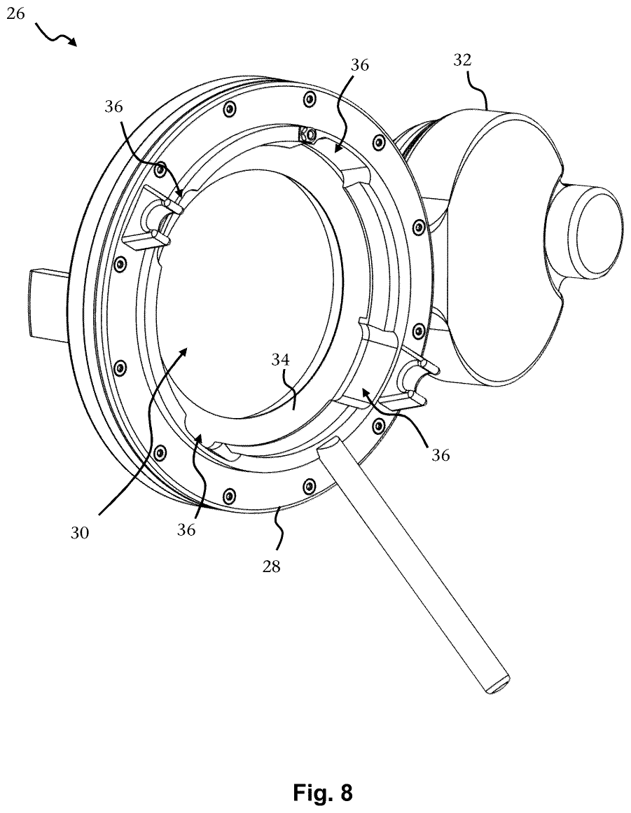

The alpha port 26 is detailed in FIGS. 7 and 8.

The alpha port 26 comprises an alpha port base body 28. The alpha port base body 28 can be ring-shaped. Specifically, the alpha port base body 28 can extend through the isolator wall. The alpha port base body 28 has an inner side and an outer side. The inner side faces the internal space, while the outer side faces the external environment 15. In the coupled state, the outer side is particularly oriented towards the beta container 38.

The alpha port 26 comprises an alpha port opening 30. The alpha port opening 30 forms a passage through the isolator wall. The alpha port opening 30 is circular. The alpha port opening 30 can be formed by a recess in the alpha port base body 28, which extends from the inner side to the outer side of the alpha port base body 28. The alpha port base body 28 surrounds the alpha port opening 30.

The alpha port 26 further comprises a door 32. The door 32 is arranged at the alpha port opening 30. The door 32 serves to open and close the alpha port opening 30. The door 32 is movably, in particular pivotably, arranged on the alpha port base body 28. The door 32 can be moved to open or close the alpha port opening 30. In a closed state, the door 32 tightly seals the alpha port opening 30. To move the door, the alpha port 26 can, for example, comprise a drive mechanism (not shown).

The alpha port 26 comprises an alpha port flange 34. The alpha port flange 34 is arranged on the outer side of the alpha port base body 28. Specifically, the alpha port base body 28 can form the alpha port flange 34 on the outer side. The alpha port flange 34 surrounds the alpha port opening 30 on the outer side.

The alpha port 26 can further comprise one or more coupling elements 36. The coupling elements 36 serve to couple the alpha port 26 with the beta container 38. The coupling elements 36 are arranged on the alpha port base body 28, in particular on the alpha port flange 34.

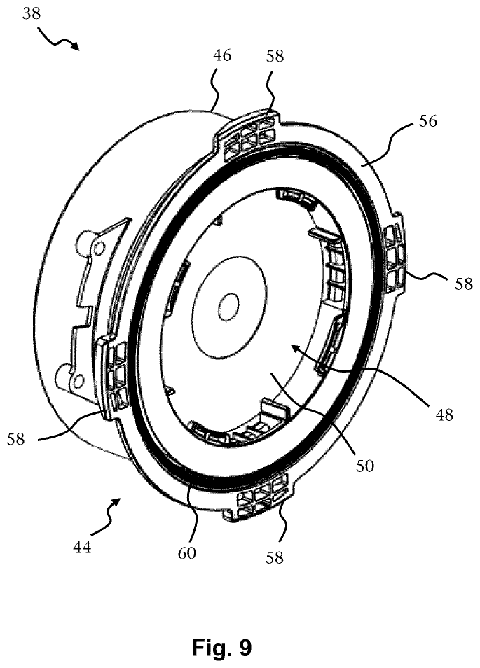

The beta container 38 is detailed in FIGS. 9 to 11.

The beta container 38 comprises an outer wall 40. The outer wall 40 can, for example, be designed to be flexible. Specifically, the outer wall 40 can be a flexible bag. The flexible bag can be made of plastic. The flexible bag can, for example, be a sterile bag. Alternatively, the outer wall 40 can be rigid. In particular, the outer wall 40 can be designed as a rigid housing. The housing can be made of plastic or metal, such as aluminum or stainless steel.

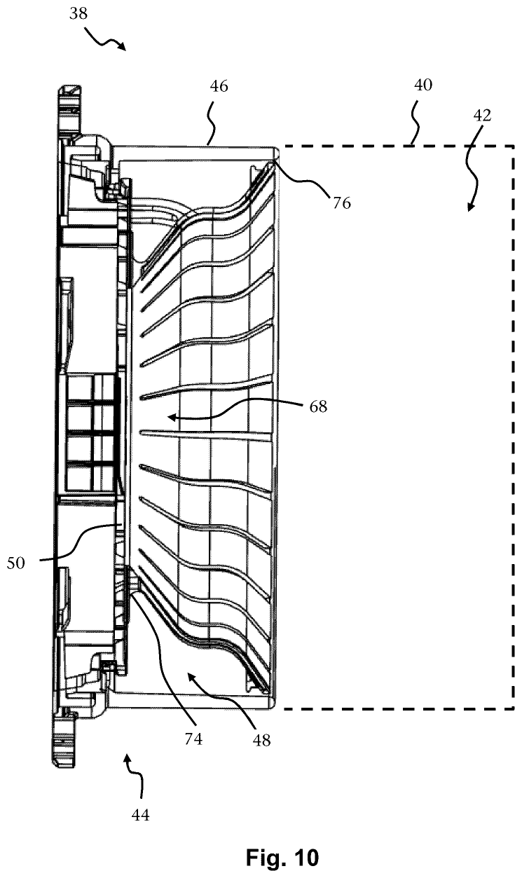

The beta container 38 further comprises an internal volume 42. Objects, such as closure elements, can be arranged in the internal volume 42. The outer wall 40 surrounds or encloses the internal volume 42. In particular, the outer wall 40 isolates the internal volume 42 from the external environment 15 surrounding the beta container 38. The outer wall 40 and the internal volume 42 are schematically illustrated in FIG. 10. In the other figures of the isolator system 10 of the first embodiment, the outer wall 40 and the internal volume 42 are not shown for illustrative purposes.

The beta container 38 comprises a beta port 44. The beta port 44 can be arranged on the outer wall 40 of the beta container 38. The alpha port 26 and the beta port 44 are couplable with each other. Specifically, the beta port 44 can be coupled to the alpha port 26 from the external environment 15. In a coupled state of the alpha port 26 and the beta port 44, the internal volume 42 of the beta container 38 is connected to the internal space 14 of the isolator 12.

The beta port 44 comprises a beta port base body 46. The beta port base body 46 is preferably ring-shaped. Specifically, the beta port base body 46 can extend through the outer wall 40. The beta port base body 46 has an inner side and an outer side. The inner side faces the internal volume 42. The outer side faces the external environment 15. In the coupled state, the outer side is particularly oriented towards the alpha port 26.

The beta port 44 comprises a beta port opening 48. The beta port opening 48 forms a passage through the outer wall 40. In other words, the internal volume 42 is accessible from the outside through the beta port opening 48. The beta port opening 48 is circular. The beta port opening 48 can be formed by a recess in the beta port base body 46, which extends from the inner side to the outer side of the beta port base body 46. Specifically, the beta port base body 46 surrounds the beta port opening 48. In the coupled state, the alpha port opening 30 and the beta port opening 48 are arranged concentrically to each other.

The beta port opening 48 and the alpha port opening 30 are essentially of the same size. Specifically, the beta port opening 48 and the alpha port opening 30 can have the same diameter.

The beta port 44 comprises a beta port flange 56. The beta port flange 56 is arranged on the outer side of the beta port base body 46. Specifically, the beta port base body 46 can form the beta port flange 56 on the outer side. The beta port flange 56 surrounds the beta port opening 48 on the outer side. In the coupled state, the alpha port flange 34 and the beta port flange 56 rest circumferentially against each other.

The beta port 44 can further comprise a sealing element 60. The sealing element 60 serves to seal the coupling between the alpha port 26 and the beta port 44. The sealing element 60 can be arranged on the outer side of the beta port base body 46. Specifically, the sealing element 60 can be arranged on the beta port flange 56. In particular, the beta port flange 56 can have a circumferential groove on the outer side around the beta port opening 48, into which the sealing element 60 is inserted. The sealing element 60 can, for example, be a lip seal. In the coupled state, the sealing element 60 is arranged between the alpha port flange 34 and the beta port flange 56. In this way, the internal volume 42 of the beta container 38 and the internal space 14 of the isolator 12 are sealed against the external environment 15.

The beta port 44 comprises a cover element 50. The cover element 50 is arrangeable at or in the beta port opening 48. The cover element 50 serves to cover or seal the beta port opening 48.

When the cover element 50 is arranged at or in the beta port opening 48 and covers the beta port opening 48, the beta container 38 is closed. The beta container 38 is then in a closed state. In FIG. 9, the beta container 38 is shown in the closed state.

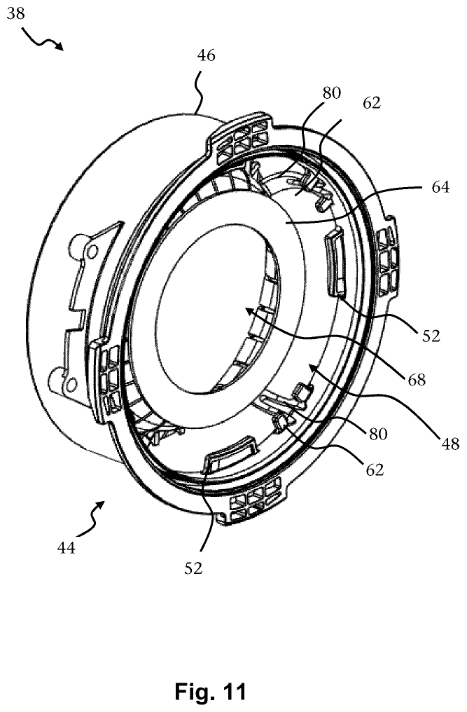

When the cover element 50 is not arranged at or in the beta port opening 48 and does not cover the beta port opening 48, in particular when the cover element 50 is removed from the beta port opening 48, the beta container 38 is open. The beta container 38 is then in an open state. In FIG. 11, the beta container 38 is shown in the open state without the cover element 50.

The cover element 50 can be detachably couplable with the beta port base body 46 or the beta port flange 56. When the cover element 50 is coupled with the beta port base body 46 or the beta port flange 56, the cover element 50 seals the beta port opening 48. For coupling, the cover element 50 and the beta port base body 46 can comprise corresponding coupling elements. Specifically, the beta port base body 46 can comprise protrusions 52 in the beta port opening 48, and the cover element can have corresponding recesses 54 for the protrusions 52. To fasten the cover element 50 at or in the beta port opening 48, the protrusions 52 can be engaged with the recesses.

The beta port 44 can further comprise one or more coupling elements 58. The coupling elements 58 serve to couple the beta port 44 with the alpha port 26. Specifically, the coupling elements 58 of the beta port can couple with the coupling elements 36 of the alpha port to connect the alpha port and the beta port. The coupling elements 58 are arranged on the beta port base body 46, in particular on the beta port flange 56.

The door 32 and the cover element 50 are couplable with each other. When the door 32 and the cover element 50 are coupled, they can be moved together. In particular, they can be moved in such a way that the alpha port opening 30 and the beta port opening 48 can be opened and closed together. Specifically, the door 32 and the cover element 50 can couple with each other when the alpha port 26 and the beta port 44 are coupled. As a result, the alpha port opening 30 and the beta port opening 48 can be opened and closed together when the alpha port 26 and the beta port 44 are coupled.

For coupling, the door 32 and the cover element 50 can comprise corresponding coupling elements, wherein one or more coupling elements of the door 32 are couplable with one or more coupling elements of the cover element 50.

Objects, such as closure elements, can be provided in the beta container 38, which are to be transferred into the isolator 12. In the coupled state of the alpha port 26 and the beta port 44, the objects can then be transferred or introduced into the isolator 12. Specifically, the objects can be supplied from the beta container through the beta port 44, the alpha port 26, and the feed device 16 into the isolator.

The beta container 38 further comprises a functional element 64. The functional element serves to guide the transfer of objects between the beta container and the isolator. The functional element 64 is preferably arrangeable in the beta container 38. The functional element 64 is arrangeable on and/or in the beta port opening 48. Preferably, the functional element 64 can be at least partially arranged in the beta port opening 48. The functional element is particularly arrangeable on the beta port base body 46.

In this embodiment, the functional element 64 is designed as an insert element that is insertable into the beta container 38, in particular into the beta port opening 48. Specifically, the functional element 64 can be fastened, preferably detachably, to the beta port base body 46. In the inserted state, the functional element 64 is fixed to the beta port base body.

In the closed state of the beta container 38, the functional element 64 can be arranged between the cover element 50 and the internal volume 42. Specifically, the functional element 64 is arranged on or adjacent to the cover element 50.

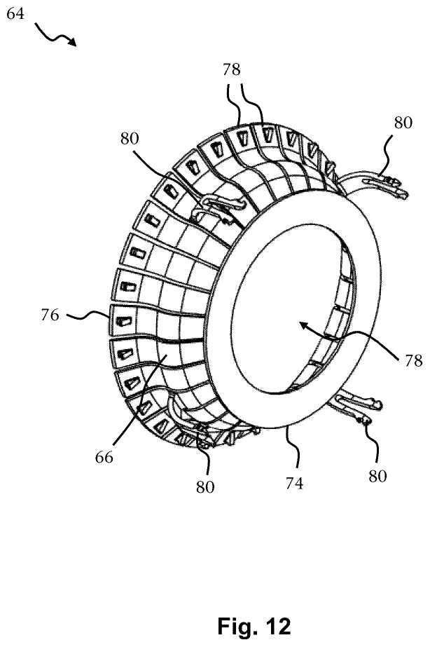

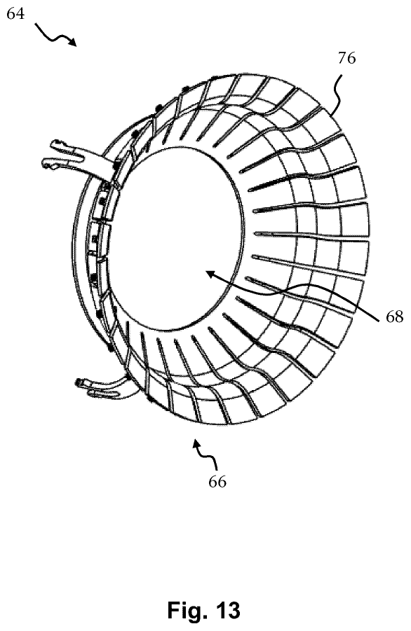

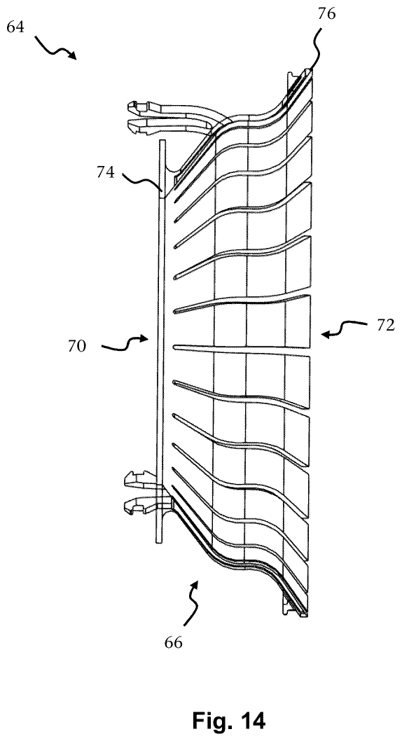

The functional element 64 is detailed in FIGS. 12 to 14.

The functional element 64 comprises a functional element body 66. The functional element body 66 comprises an outer edge 76. The outer edge 76 is arranged on the beta port base body. Specifically, the outer edge 76 rests circumferentially against the beta port base body 46 in the beta port opening 48 when the functional element 64 is at least partially arranged or inserted in the beta port opening 48. The outer edge 76 is preferably shaped complementary to the beta port opening 48. Specifically, the outer edge 76 can be arranged circumferentially in the beta port opening 48 on the beta port base body 46 or rest circumferentially against it.

The functional element 64 comprises a functional element opening 68. The functional element opening 68 is circular. The functional element opening 68 is smaller than the beta port opening 48. Specifically, the diameter of the functional element opening 68 is smaller than the diameter of the beta port opening 48. The functional element opening 68 is arrangeable concentrically to the beta port opening 48. Specifically, in the inserted state, the functional element opening 68 is arranged concentrically to the beta port opening 48.

The functional element opening 68 is designed as a recess in the functional element body 66. The functional element opening 68 extends in an axial direction through the functional element body 66. The functional element body 66 surrounds the functional element opening 68. The functional element body 66 extends radially outward from the functional element opening 68. The functional element body 66 is preferably ring-shaped or funnel-shaped.

The functional element 64 extends in the axial direction from a first axial end 70 to a second axial end 72. In the inserted state, the second axial end 72 faces the internal volume 42, and the first axial end 70 faces away from the internal volume 42. When the beta port 44 is coupled with the alpha port 26, the first axial end 70 is oriented towards the alpha port 26, and the second axial end 72 is oriented away from the alpha port 26.

The functional element 64 comprises a functional element flange 74. The functional element body comprises the functional element flange 74 at the first axial end 70. The functional element flange 74 surrounds the functional element opening 68 at the first axial end 70. In the inserted state, the functional element 64 is arranged in the beta port opening 48 in such a way that the functional element flange 74 is positioned adjacent to the cover element 50 when the beta port opening is covered or sealed with the cover element 50.

The outer edge 76 is arranged at the second axial end 72. The outer edge 76 is positioned radially outward. Specifically, the outer edge 76 is arranged further radially outward than the functional element flange 74.

The outer edge 76 is elastically formed, in particular in a lamellar manner. For this purpose, the functional element body 66 can comprise a plurality of lamellae circumferentially arranged, each extending up to the outer edge 76.

The functional element 64 can comprise one or more fastening elements 80, by means of which the functional element 64 can be fastened to the beta port. The fastening elements 80 can be hook-shaped. Preferably, the fastening elements 80 are arranged on the outer edge 76 of the functional element body 66. The beta port 44 can comprise one or more corresponding receiving elements 62. The receiving elements 62 are arranged on the beta port base body 46 in the beta port opening 48. The fastening elements 80 can engage with the receiving elements 62 to secure the functional element 64. Each fastening element 80 can engage with a corresponding receiving element 62.

As previously described, objects such as closure elements can be provided in the internal volume 42 of the beta container 38, and these objects can then be introduced or supplied into the internal space of the isolator 12 using the transfer system 24 and the feed device 16. For this purpose, the beta port 44 is first coupled with the alpha port 26, and the door 32 is opened together with the cover element 50. Then, the tube 18 of the feed device 16 can be pivoted into the first position. This state is shown in FIGS. 1 and 2. The objects can then be transferred from the internal volume 42 through the beta port opening 48, the alpha port opening 30, and the tube 18 into the isolator 12. The functional element 64 is arranged in such a way that the objects must pass through the functional element opening 68 when passing through the beta port opening 48.

Since the alpha port 26 is arranged on a vertical wall surface of the isolator wall, the alpha port opening 30 extends in a horizontal direction from the inner side to the outer side of the alpha port base body 28. The tube 18 is arranged at an angle to the horizontal direction in the first position. As a result, the tube 18 is aligned at an angle to the alpha port opening 30 in the first position.

In the first position of the tube 18, the first end 82 of the tube extends into the alpha port opening 30 up to the beta port opening 48. Specifically, the first end 82 of the tube extends up to the functional element flange 74. The first end 82 of the tube rests against the functional element flange 74. In particular, the functional element opening 68 is designed to be smaller than or equal to the opening of the tube 18 at the first end 82. The functional element opening 68 is aligned with the opening of the tube 18 at the first end 82. As a result, all objects exiting the functional element opening 68 at the first axial end 70 enter directly into the tube 18.

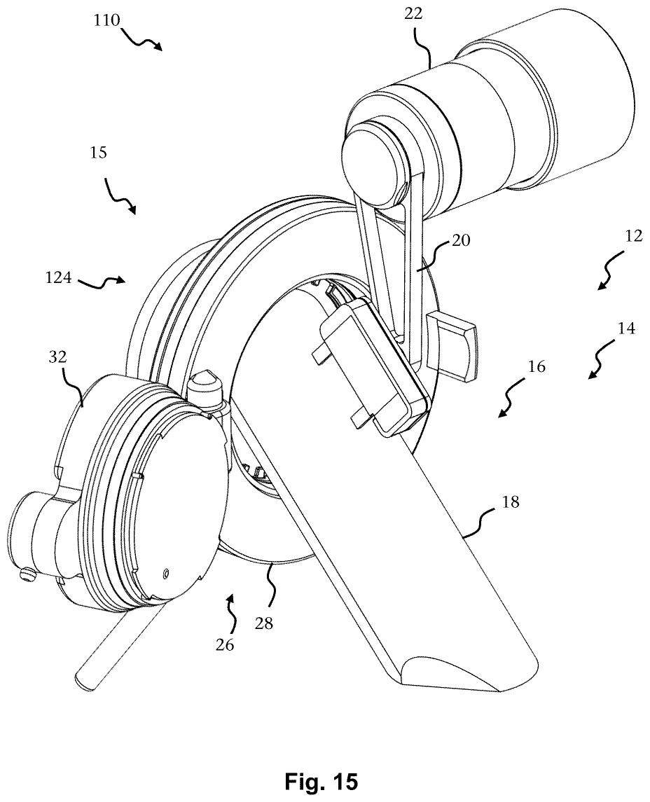

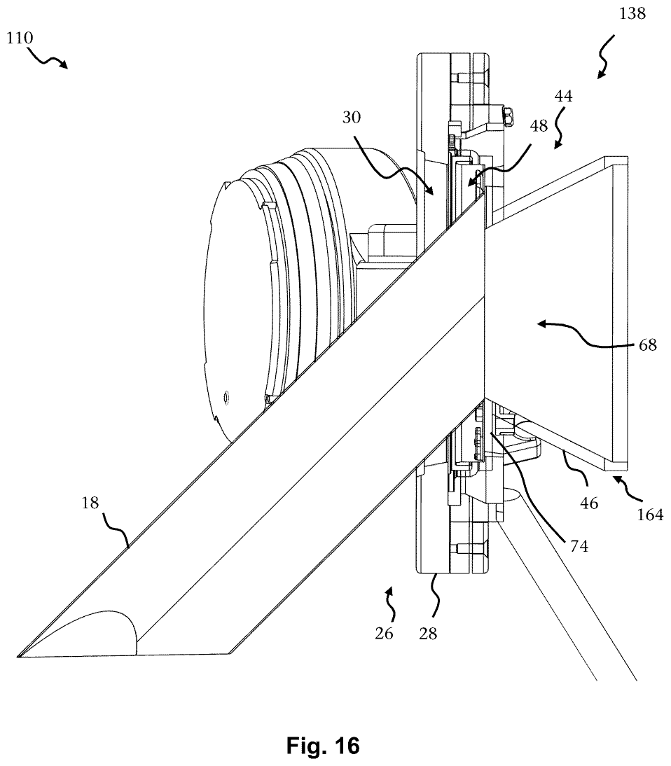

FIGS. 15 and 16 show a second embodiment of an isolator system, designated as barrier system 110 in its entirety. The isolator system 110 of the second embodiment essentially corresponds to the isolator system 10 of the first embodiment. Identical elements are designated with the same reference numerals and will not be further explained below. The isolator system 110 differs from the isolator system 10 in the design of the transfer system.

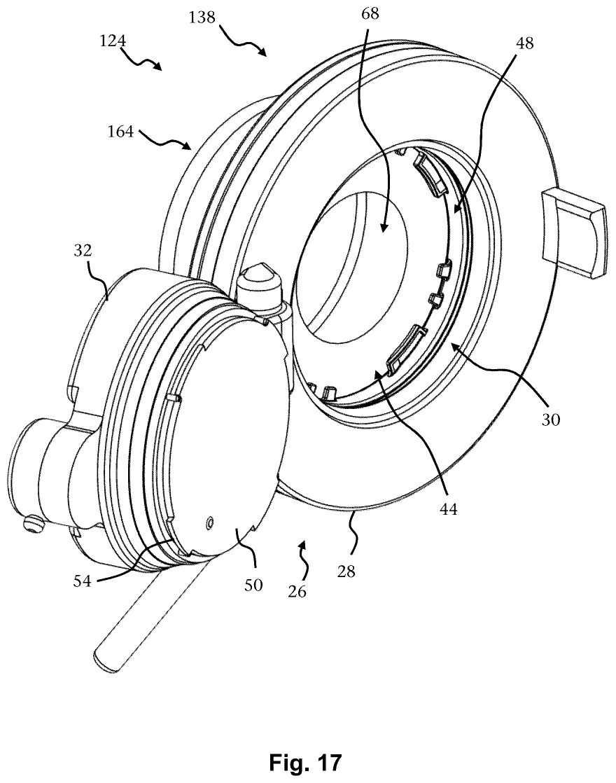

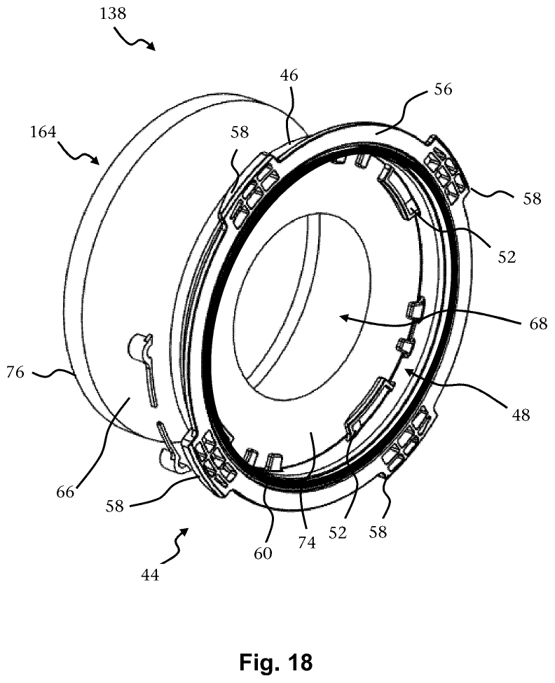

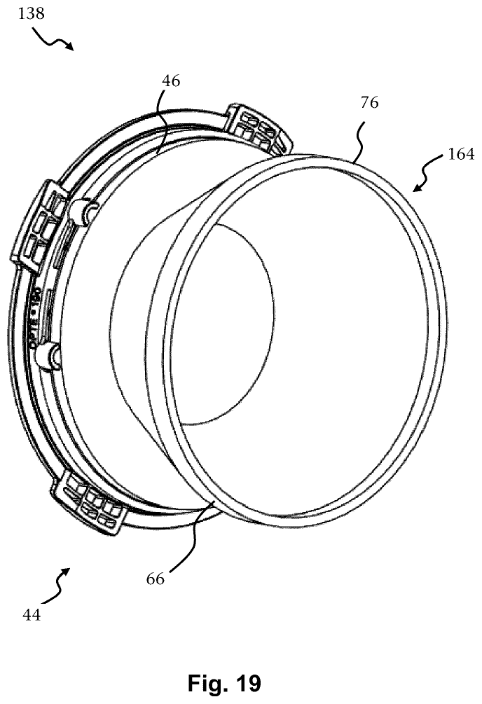

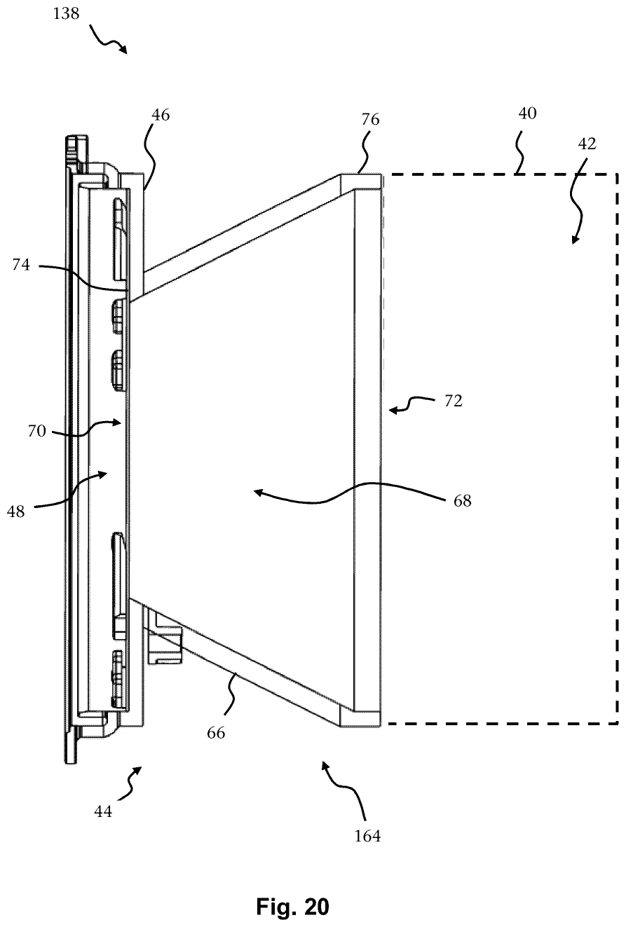

The transfer system of the isolator system 110 is designated with reference numeral 124. The transfer system 124 is shown in FIG. 17. The transfer system 124 comprises the same alpha port 26 as the transfer system 24 of the isolator system 10. The transfer system 124 further comprises a beta container 138. The beta container 138 has essentially the same structure as the beta container 38. The beta container 138 differs from the beta container 38 in the design of the functional element. The beta container 138 is shown in FIGS. 18 to 20.

The functional element of the beta container 138 is designated with reference numeral 164. The functional element 164 is integrally formed with the beta port base body 46. Specifically, in this embodiment, the functional element 164 is not designed as an insert element. Accordingly, the beta port does not comprise receiving elements 62, and the functional element 164 does not comprise fastening elements 80.

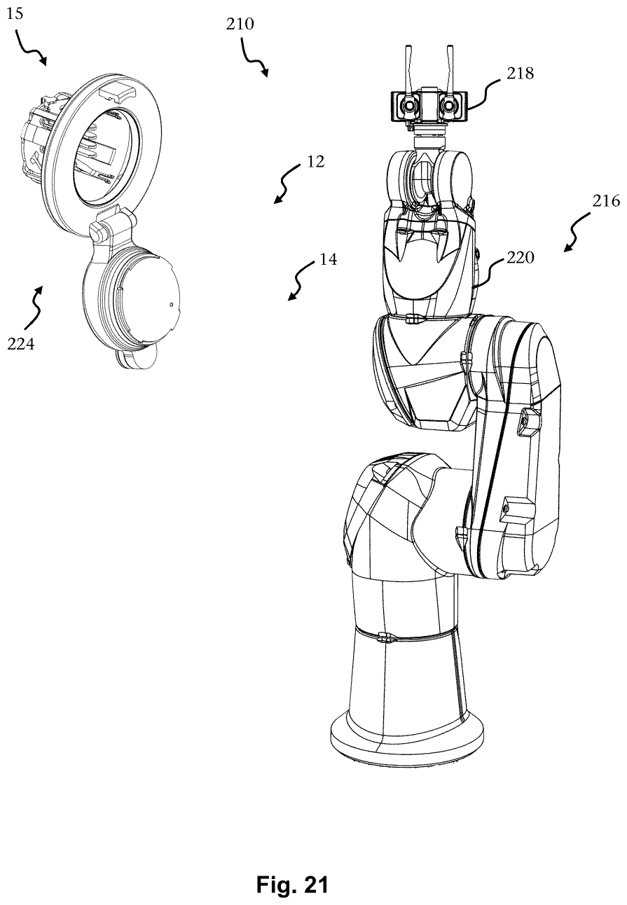

FIG. 21 shows a third embodiment of an isolator system, designated as barrier system 210 in its entirety. The isolator system 210 of the third embodiment has a similar structure to the isolator system 10 of the first embodiment. Identical elements are designated with the same reference numerals and will not be further explained below.

In the third embodiment, the isolator system 210, in the depicted form, does not include a feed device 16.

The isolator system 210 comprises a handling device 216 inside the isolator 12. The handling device 216 is used for handling, in particular transferring, nutrient medium carriers. The handling device 216 comprises an end effector 218. The end effector 218 can be a gripping tool, such as a gripper. The gripping tool can be used to grasp and hold nutrient medium carriers. The handling device 216 comprises a multi-axis arm 220. The end effector 218 is arranged at one end of the arm 220. The end effector 218 can be moved within the isolator 12 using the arm 220. To move the arm, the handling device 216 can, for example, comprise a drive mechanism.

The isolator system 210 of the third embodiment further differs from the isolator system 10 of the first embodiment in the design of the transfer system.

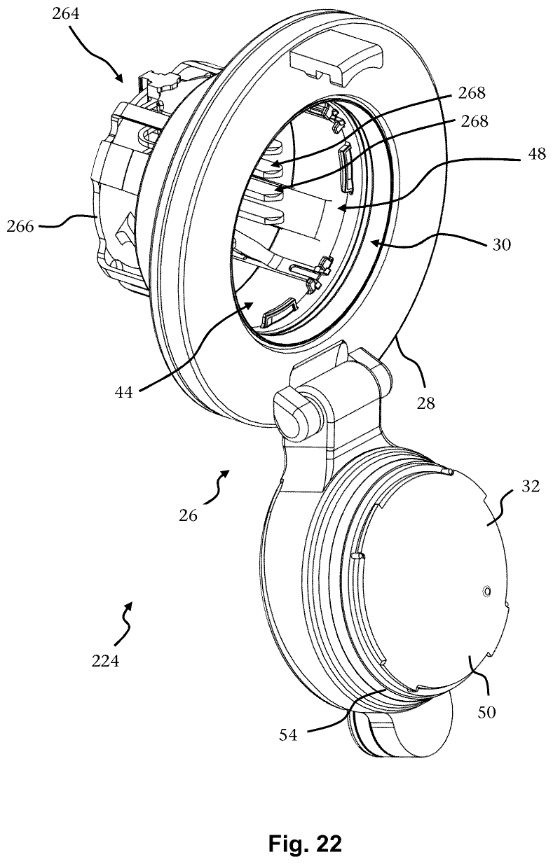

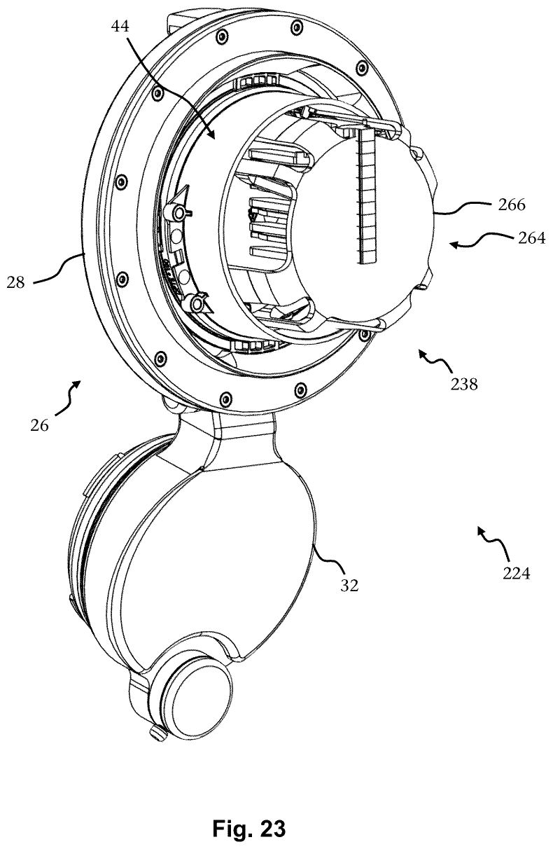

The transfer system of the isolator system 210 is designated with reference numeral 224. The transfer system 224 is shown in FIGS. 22 and 23. The transfer system 224 serves to transfer nutrient medium carriers into or out of the isolator.

The transfer system 224 comprises the same alpha port 26 as the transfer system 24 of the isolator system 10. In the transfer system 224, the alpha port 26 is arranged in a rotated position so that the door 32 of the alpha port 26 is not positioned laterally next to the alpha port opening 30 on the alpha port base body 28, but below the alpha port opening 30.

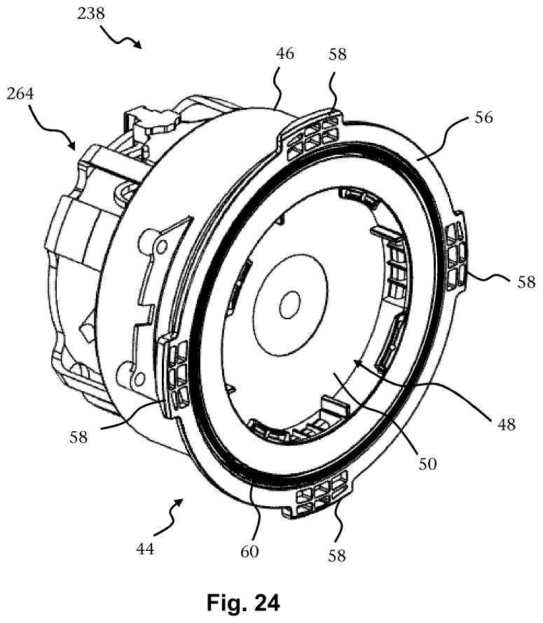

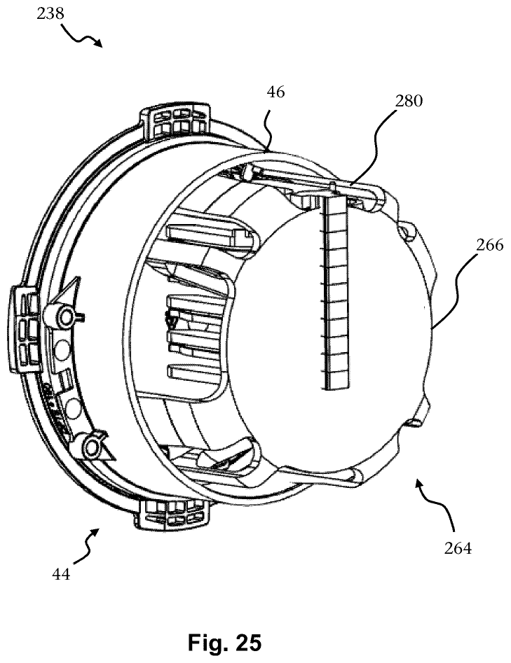

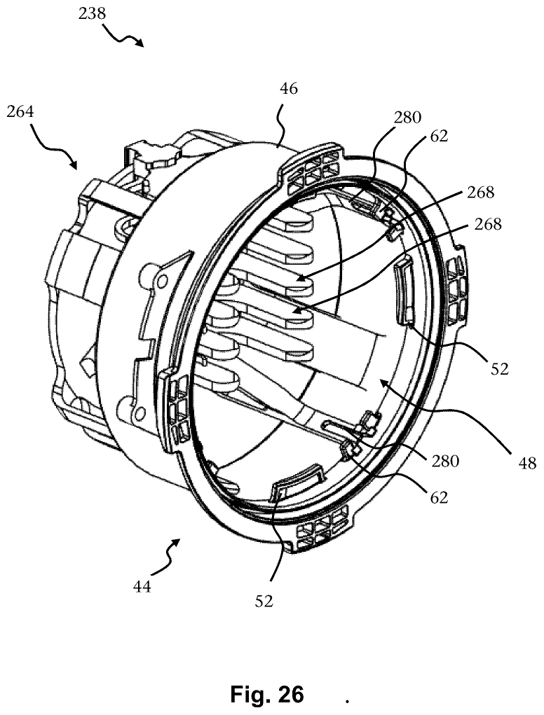

The transfer system 224 further comprises a beta container 238. The beta container 238 has essentially the same structure as the beta container 38. The beta container 238 differs from the beta container 38 in the design of the functional element. The beta container 238 is shown in FIGS. 24 to 26. In FIG. 24, the beta container 238 is shown in the closed state. In FIG. 26, the beta container 238 is shown in the open state without the cover element 50.

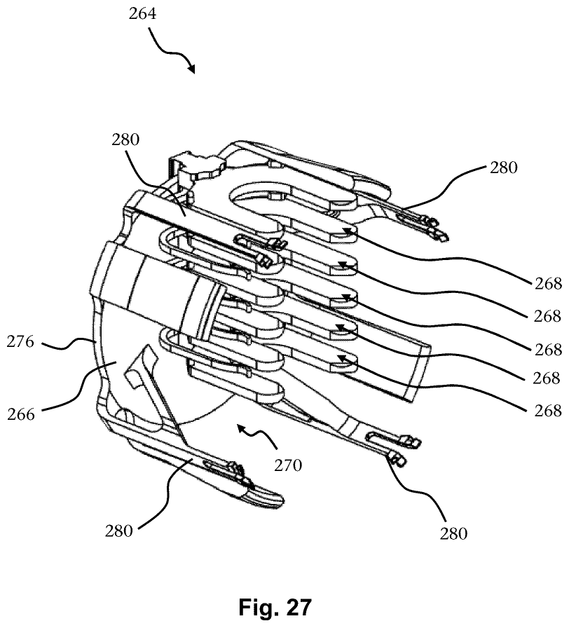

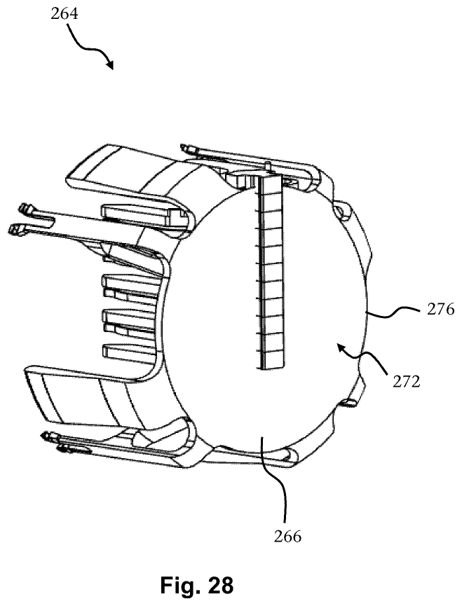

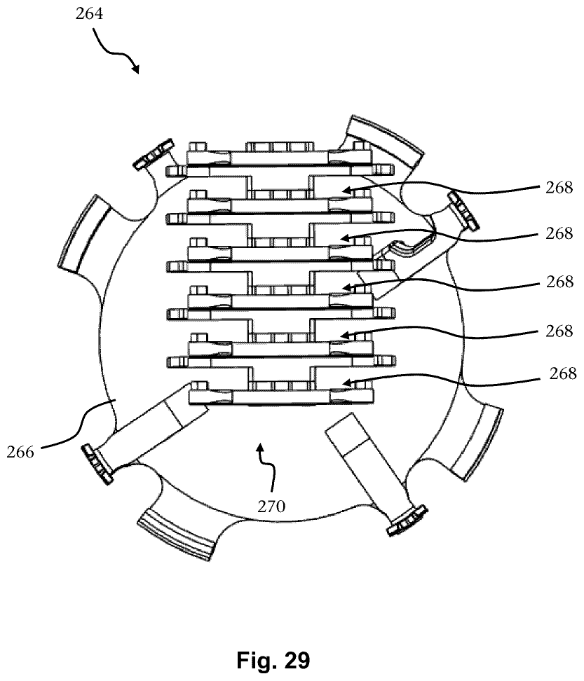

The functional element of the beta container 238 is designated with reference numeral 264. The functional element 264 has a different structure than the functional element 38 of the first embodiment. The functional element 264 is shown in FIGS. 27 to 29. The functional element 264 serves to transfer one or more nutrient medium carriers into or out of the isolator.

The functional element 264 is preferably arrangeable in the beta container 238. The functional element 264 is particularly arrangeable on and/or in the beta port opening 48. In the illustrated embodiment, the functional element 264 is at least partially arrangeable in the beta port opening 48.

In this embodiment, the functional element 264 is designed as an insert element that is insertable into the beta container 238, in particular into the beta port opening 48. Specifically, the functional element 264 can be fastened, preferably detachably, to the beta port base body 46. Specifically, the functional element 264 is fixed to the beta port base body 46 in the inserted state.

In the closed state of the beta container 238, the functional element 264 can be arrangeable between the cover element 50 and the internal volume 42. Specifically, the functional element 264 can be arranged in the internal volume 42 and extend from the internal volume 42 into the beta port opening 48.

The functional element 264 comprises a functional element body 266. The functional element 264 further comprises one or more nutrient medium carrier holders 268. The one or more nutrient medium carrier holders 268 are arranged on the functional element body 266. Specifically, the nutrient medium carrier holders 268 are attached to or formed by the functional element body 266.

Each nutrient medium carrier holder 268 is configured to hold a nutrient medium carrier. Specifically, each nutrient medium carrier holder 268 can accommodate a nutrient medium carrier. Each nutrient medium carrier holder can, for example, comprise a receiving element or a support surface for a respective nutrient medium carrier.

The functional element body 266 can be essentially disk-shaped. The functional element body 266 comprises a first side 270 and an opposite second side 272. The nutrient medium carrier holders 268 are arranged on the first side 270 of the functional element body 266. The functional element body 266 comprises an outer circumferential edge 276.

In the inserted state, the functional element body 266 is arranged in the internal volume 42. In the inserted state, the nutrient medium carrier holders 268 extend from the functional element body 266 into the beta port opening 48. The first side 270 faces the beta port opening 48 in the inserted state. The second side 272 faces away from the beta port opening 48 in the inserted state. In the coupled state of the alpha port 26 and the beta port 44, the first side 270 is therefore oriented towards the alpha port 26.

The functional element 264 can comprise one or more fastening elements 280, by means of which the functional element 264 can be fastened to the beta port 44. The fastening elements 280 can be hook-shaped. Preferably, the fastening elements 280 are arranged on the outer edge 276 of the functional element body 266. The fastening elements 280 can engage with the receiving elements 62 to secure the functional element 264. Each fastening element 280 can engage with a corresponding receiving element 62.

To transfer nutrient medium carriers into the isolator 12, the nutrient medium carriers can first be provided in the nutrient medium carrier holders 268 in the beta container 238. Then, the beta port 44 can be coupled with the alpha port 26, and the door 32 can be opened together with the cover element 50. The handling device 216 can then individually grasp the nutrient medium carriers and transfer them from the beta container 238 into the isolator 12, preferably to a defined position within the isolator 12.

The transfer system 224 can also be used to transfer nutrient medium carriers out of the isolator 12. When the beta port 44 is coupled with the alpha port 26 and the door 32 is opened together with the cover element 50, the handling device 216 can individually transfer the nutrient medium carriers from the isolator 12 into the beta container 238 and place them in one of the nutrient medium carrier holders 268. The beta port 44 and the alpha port 26 can then be closed and decoupled.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A functional element for a beta container of a transfer system, wherein the functional element is arrangeable on and/or in a beta port opening of a beta port of the beta container, the functional element comprises a functional element opening, and the functional element opening is smaller than the beta port opening.

2. The functional element according to claim 1, wherein the functional element opening is circular.

3. The functional element according to claim 1, wherein the functional element opening is arrangeable concentrically to the beta port opening.

4. The functional element according to claim 1, wherein the functional element is ring-shaped or funnel-shaped.

5. The functional element according to claim 1, wherein the functional element is designed as an insert element that is insertable into the beta container.

6. The functional element according to claim 1, wherein the functional element comprises a functional element body, the functional element body surrounds the functional element opening or one or more nutrient medium carrier holders are arranged on the functional element body.

7. The functional element according to claim 6, wherein the functional element body comprises an outer edge, the outer edge is elastically formed.

8. The functional element according to claim 6, wherein the functional element body comprises an outer edge, the outer edge is formed complementary to the beta port opening.

9. The functional element according to claim 1, wherein the functional element comprises one or more fastening elements, by means of which the functional element is attachable to the beta port.

10. A functional element for a beta container of a transfer system, wherein the functional element is arrangeable on and/or in a beta port opening of a beta port of the beta container, the functional element comprises one or more nutrient medium carrier holders, and each nutrient medium carrier holder is configured to hold a nutrient medium carrier.

11. A beta container for a transfer system, wherein the beta container comprises an internal volume, an outer wall enclosing the internal volume, a beta port that is couplable with an alpha port of the transfer system, and a functional element for the beta container of the transfer system, the functional element is arrangeable on and/or in a beta port opening of the beta port of the beta container, the functional element comprises a functional element opening, and the functional element opening is smaller than the beta port opening.

12. The beta container according to claim 11, wherein the beta port comprises the functional element, and a beta port base body of the beta port and the functional element are integrally formed.

13. The beta container according to claim 11, wherein the beta port comprises a cover element for covering the beta port opening, and the functional element is arranged between the cover element and the internal volume.

14. The beta container according to claim 11, wherein the beta port comprises a sealing element for sealing the coupling between the alpha port and the beta port.

15. A beta container for a transfer system, wherein the beta container comprises an internal volume, an outer wall enclosing the internal volume, a beta port that is couplable with an alpha port of the transfer system, and a functional element for the beta container of the transfer system, the functional element is arrangeable on and/or in a beta port opening of the beta port of the beta container, the functional element comprises one or more nutrient medium carrier holders, and each nutrient medium carrier holder is configured to hold a nutrient medium carrier.

16. The beta container according to claim 15, wherein the beta port comprises the functional element, and a beta port base body of the beta port and the functional element are integrally formed.

17. The beta container according to claim 15, wherein the beta port comprises a cover element for covering the beta port opening, and the functional element is arranged between the cover element and the internal volume.

18. The beta container according to claim 15, wherein the beta port comprises a sealing element for sealing the coupling between the alpha port and the beta port.

19. A barrier system, wherein the barrier system comprises a transfer system comprising an alpha port and a beta container, wherein the beta container comprises an internal volume, an outer wall enclosing the internal volume, a beta port that is couplable with the alpha port of the transfer system, and a functional element for the beta container of the transfer system, the functional element is arrangeable on and/or in a beta port opening of the beta port of the beta container, the functional element comprises a functional element opening, and the functional element opening is smaller than the beta port opening.

20. The barrier system according to claim 19, wherein the barrier system comprises an isolator, the isolator comprises an internal space and an isolator wall enclosing the internal space, the alpha port is arranged on the isolator wall, and the beta port is couplable to the alpha port from an external environment of the isolator.

21. The barrier system according to claim 20, wherein the alpha port is arranged on a vertical wall surface of the isolator wall.

22. The barrier system according to claim 19, wherein the barrier system further comprises a feed device in the internal space of the isolator, and the feed device is arrangeable on the alpha port from the inside.

23. The barrier system according to claim 22, wherein the feed device comprises a tube, and a first open end of the tube is arrangeable on the alpha port.

24. The barrier system according to claim 23, wherein the tube of the feed device is arrangeable on the alpha port such that, in the coupled state, it extends through an alpha port opening of the alpha port up to the beta port opening, and the first end of the tube is arrangeable on a functional element flange of the functional element.

25. The barrier system according to claim 23, wherein the tube of the feed device is arrangeable on the alpha port such that the tube extends at an angle to a horizontal direction.

26. The barrier system according to claim 23, wherein the first end of the tube has an elliptical shape.

27. The barrier system according to claim 19, wherein the barrier system is an isolator system.

28. A barrier system, wherein the barrier system comprises a transfer system comprising an alpha port and a beta container, the beta container comprises an internal volume, an outer wall enclosing the internal volume, a beta port that is couplable with the alpha port of the transfer system, and a functional element for the beta container of the transfer system, the functional element is arrangeable on and/or in a beta port opening of the beta port of the beta container, the functional element comprises one or more nutrient medium carrier holders, and each nutrient medium carrier holder is configured to hold a nutrient medium carrier.

29. The barrier system according to claim 28, wherein the barrier system comprises an isolator, the isolator comprises an internal space and an isolator wall enclosing the internal space, the alpha port is arranged on the isolator wall, and the beta port is couplable to the alpha port from an external environment of the isolator.

30. The barrier system according to claim 29, wherein the alpha port is arranged on a vertical wall surface of the isolator wall.

31. The barrier system according to claim 28, wherein the barrier system further comprises a feed device in the internal space of the isolator, and the feed device is arrangeable on the alpha port from the inside.

32. The barrier system according to claim 31, wherein the feed device comprises a tube, and a first open end of the tube is arrangeable on the alpha port.

33. The barrier system according to claim 32, wherein the tube of the feed device is arrangeable on the alpha port such that, in the coupled state, it extends through an alpha port opening of the alpha port up to the beta port opening, and the first end of the tube is arrangeable on a functional element flange of the functional element.

34. The barrier system according to claim 32, wherein the tube of the feed device is arrangeable on the alpha port such that the tube extends at an angle to a horizontal direction.

35. The barrier system according to claim 32, wherein the first end of the tube has an elliptical shape.

36. The barrier system according to claim 28, wherein the barrier system is an isolator system.
